(19) United States
(12) Patent Application Publication
(10) Pub. No.: US 2011/0306941 A1
(43) Pub. Date: Dec. 15, 2011

(54) Portable Lavage Apparatus

(76) Inventors: N.R. Chandrasekar, Canton, MA (US); Donald McKay, Belmont, MA (US); Andrew E. Goddard, Beverly, MA (US); William G. Gnesda, Imperial Beach, CA (US)

(21) Appl. No.: 13/160,467

(22) Filed: Jun. 14, 2011

Related U.S. Application Data
(60) Provisional application No. 61/397,628, filed on Jun. 14, 2010.

(51) Int. Cl. A61M 31/00

(52) U.S. Cl. 604/262

(57) ABSTRACT

A portable lavage apparatus for irrigating or washing wounds is disclosed. The apparatus includes a fluid bag, a spring bias mechanism, and a hose. The spring bias mechanism is typically configured to store potential energy or produce kinetic energy used for urging fluid from the fluid bag, through the hose, and toward a wound. The invention has particular advantages in military or civilian environments in that it allows prompt treatment of wounds. Additionally, the lavage apparatus is small enough to be carried by individuals and does not require an external source of power. Accordingly, the portable lavage apparatus may be employed even on battle fields or in remote areas to treat wounds.

### CROSS-REFERENCE TO RELATED APPLICATIONS

This application claims the priority benefit of provisional application No. 61/397,628 filed Jun. 14, 2009, entitled “Portable Lavage Apparatus,” the disclosure of which is hereby incorporated by reference.

### BACKGROUND

1. Field of the Invention

The invention relates generally to portable medical devices, and more particularly to a medical irrigating and/or washing device.

2. Background Art

In battle, wounds to the extremities are the most common type of injury. Treatment of such wounds should begin in the field, well before professional expertise is available. Typically for every group of 10-12 soldiers, one specially trained person is available who carries a medical kit. For example, within a few hundred feet behind the front lines, triage units are set up for immediate care of wounded soldiers. In addition to wounds from a variety of weapons, soldiers may also be exposed to chemicals, biological agents, snakes, and other venomous creatures. Regardless of the source of injury, wounded soldiers all need immediate, appropriate care to minimize or prevent death and disabilities.

Historically, from the era of the Trojan War to the Persian Gulf War, injury patterns have more or less remained the same. An estimate of the mortality rate during the Trojan War era is approximately 77%. Most of these casualties may be attributed to wound shock. During European wars in the 18th and 19th century, wounded soldiers died from collapse of vital functions. Amputation was performed to prevent gangrene and death during the American Civil War (1861-1865). During World War I (1914-1918), excisional debridement replaced amputation for prevention of infection and gangrene. During World War II (1939-1945), common fatalities were classified as hemorrhagic, traumatic, and septic. Mortality decreased from 20% in World War II to 1.8% in the Vietnam War (1955-1975). This may be attributable to improvements in support care through the Korean War (1950-1953), and to the discovery of penicillin in 1928 and its clinical use in 1940. Debridement with drainage and leaving wounds open for healing occur by secondary intention. Recent data from the Persian Gulf War (1990-1991) indicate that wounds to the extremities lead to the highest disability rate. Although the mortality rates have been substantially reduced, still the morbidity and long term disabilities associated with any wound treated by debridement and drainage and leaving the wound open remain the same.

Regardless of setting (e.g., civilian or military), wounds and infections are treated in a similar manner, with antibiotics, debridement or incision, and drainage. These procedures require expensive dressing changes by trained health care personnel, and may require multiple secondary surgical procedures with potentially poor outcomes (e.g., permanent disabilities). As indicated by war-time experiences dating from ancient times to the present day, an important aspect of care is attending to serious wounds in the battle field.

When treating infected wounds, open fractures, and wounds contaminated with chemicals and dirt—similar to what soldiers may encounter in a battle field—a better outcome may be attained if the injury is treated with adequate irrigation, debridement, and immediate wound closure. Several hundred cases treated by this unconventional method demonstrate that it can be an effective procedure for caring for injuries (e.g., wounds and open fractures) and may lead to excellent results with full functional recovery (i.e., minimal or no disability). However, current medical devices do not easily attain comparable results for soldiers wounded in the field or even civilians in an optimal environment with a skilled surgeon.

Wounds are typically cleansed with saline solution, using different types of tubing and syringes. Surgeons may use a pulsed irrigator to irrigate wounds. However, current medical devices may cause harm even in the hands of a well-trained surgeon. Pulsatile flow of solution even under low pressure (e.g., low flow and speed) may cause a significant amount of trauma to tissues and vital structures such as arteries, veins, and nerves. Present medical devices may also cause trauma due to pressure on the tissues, and they may provide inadequate irrigation of the tissues, defeating the intended purpose. If wounds are left open for secondary healing to occur, further expensive care (e.g., systems of dressing changes and treatments such as wound vac (negative pressure wound therapy) frequently administered by nurses) may be required. Complications (e.g., desiccation of tissues, tendons, blood vessels, nerves) causing loss of an extremity or impaired function may also result. Pulsatile flow also depends on a battery and/or air pressured pump. Present medical devices are large (i.e., not portable) and require a fair amount of storage space.

In addition to irrigation issues and trauma caused by irrigation, the irrigated fluid—contaminated with body fluids, blood, pathogenic organisms—may not be adequately contained or disposed of safely. In the process of administering treatments such as described above, a health care provider may be exposed to dangerous contaminated fluid.

### SUMMARY OF THE INVENTION

Various embodiments of the present invention, a portable lavage apparatus, may include a case, a fluid bag, a spring bias mechanism, and a hose. The fluid bag holds a fluid, and the spring bias mechanism may apply pressure to the fluid bag to urge fluid from the bag. The apparatus may further include a tension member configured to extend or compress springs in the spring bias mechanism.

Other embodiments of the present invention may include a fluid bag, a bias mechanism, and a hose. A base plate may be coupled to an upper plate by a hinge. When the upper plate is pushed toward the base plate by the bias mechanism, the fluid bag is compressed to urge fluid through the hose.

### BRIEF DESCRIPTION OF THE DRAWINGS

FIG. 1 is a diagram showing an embodiment of the portable lavage apparatus in a closed position.

FIG. 2 shows the lavage apparatus in an open position.

FIG. 3 shows the lavage apparatus in an open position in readiness for use.

FIG. 4 shows the lavage apparatus in a closed position.

FIG. 5 is a perspective view of an embodiment of the portable lavage apparatus.

FIG. 6 is a perspective view of another embodiment of the portable lavage apparatus.

FIG. 7 is a perspective view of still another embodiment of the portable lavage apparatus.

FIG. 8 is a perspective view of a further embodiment of the portable lavage apparatus.

FIGS. 9A-B illustrate a still further embodiment of the portable lavage apparatus, depicting it in both relaxed and compressed positions.

FIG. 10 is a fragmentary side elevation view of the apparatus of FIG. 9.

FIG. 11 is a schematic perspective view of the apparatus of FIG. 9.

FIG. 12 is a perspective view of another embodiment of the portable apparatus of the present invention.

FIG. 13 is a perspective view of still another embodiment of the lavage apparatus of the present invention.

FIGS. 14A-C show the device used as an IV apparatus.

### DETAILED DESCRIPTION

A medical device according to various embodiments of the present invention may include (but not be limited to) the following advantageous features: portability, consumes minimal storage space, may operate without an external source of power source (e.g., by using batteries and pneumatic power), substantial portion of device is biodegradable, can be used by lay person without significant medical training (i.e., easy to operate), avoids harm (e.g., trauma from suction and irrigation, traction injury, and pressure injection) caused by current medical devices, variable speed and flow control, may be used on most (if not all) body parts, and works with spray shields and fluid disposal systems (e.g., a universal spray shield with expandable chamber for safe collection and disposal of contaminated fluids).

For wounds treated according to the present invention, a patient's (e.g., soldier or civilian) skin may be immediately closed, and healing begun in a triage unit, in the battle field, or in the wilderness or other remote area. Patients may potentially return full time to their original employment with no loss of function or deficits. A patient receives immediate care by irrigating his or her wounds, where adequate irrigation may be used to avoid and prevent infection. After adequate irrigation, hemostasis with appropriate agents or mechanical devices (e.g., clips or sutures) may be applied and the skin may be closed, protecting vital structures. Patients can then be transferred to a specialty medical facility (e.g., hospital or clinic) for further care.

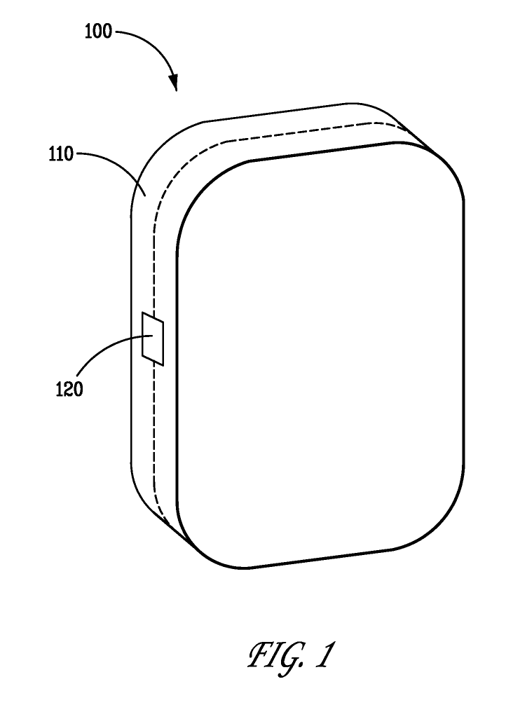

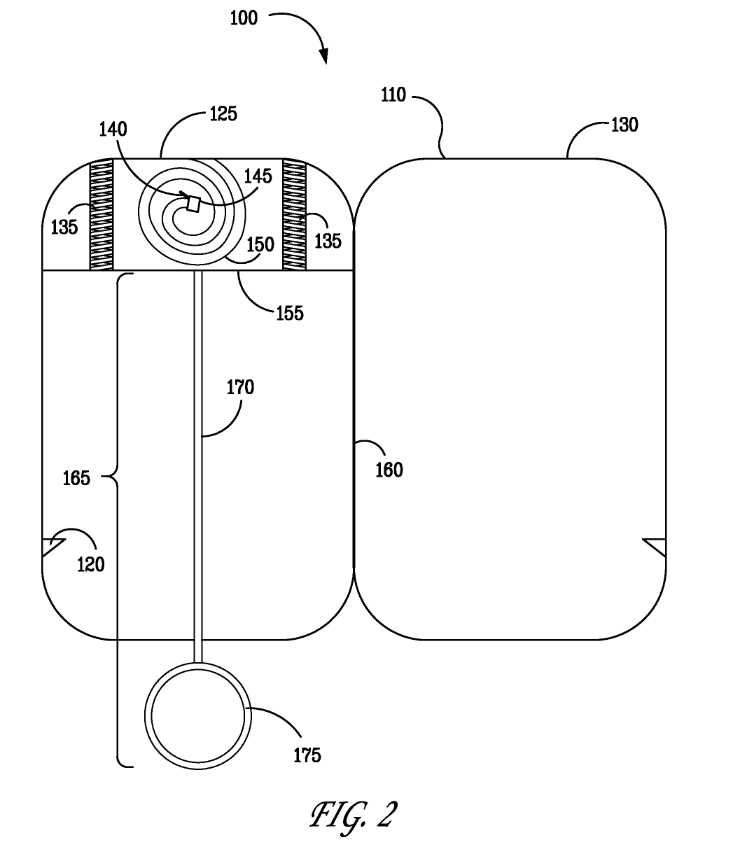

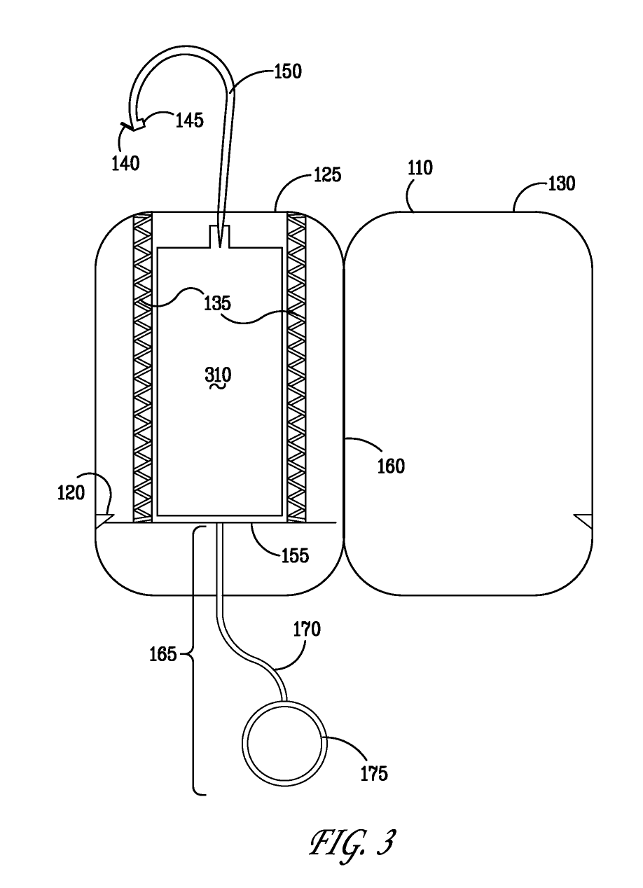

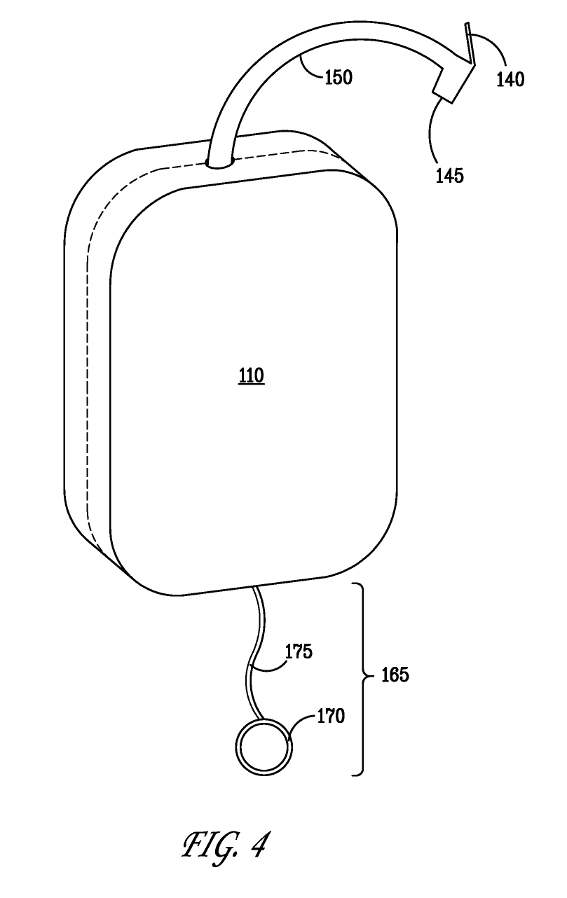

The portable lavage apparatus 100 according to embodiments of the present invention is illustrated in FIGS. 1-5. Referring first to FIG. 1, portable lavage apparatus 100 may include a case (or housing) 110. Case 110 may include two halves 125 and 130 (see FIGS. 2 and 3). Halves 125 and 130 may be coupled to each other by a hinge 155, and may be secured by a latch 120. When latch 120 secures halves 125 and 130 together, case 110 is closed as depicted in FIGS. 1 and 4. When latch 120 does not couple halves 125 and 130 together and halves 125 and 130 are positioned side-by-side, case 110 is open as shown in FIGS. 2 and 3.

Case 110 surrounds a fluid bag 310 (see FIG. 3) with a coupled dispensing hose 150. The case 110 may include a spring bias or tension mechanism which urges fluid in fluid bag 310 out through the dispensing hose 150 to outlet nozzle 145. Outlet nozzle 145 may include a finger- or thumb-operated lever 140 which controls flow of the fluid from fluid bag 310. For example, the finger- or thumb-operated lever 140 may be coupled to a valve (not shown) in outlet nozzle 145. FIG. 2 illustrates hose 150 in a coiled position within half 125 of case 110. FIG. 3 illustrates hose 150 extended out of case 110.

The spring bias mechanism, as illustrated in FIGS. 2 and 3, includes pressure plate 155. One or more springs 135 may be disposed in case 110. The springs 135 may be connected at a first end to pressure plate 155 and at a second end to case 110. As depicted in FIG. 2, pressure plate 155 may be in a rest position when springs 135 are at rest, i.e. not extended or compressed. The spring bias mechanism may also include a tension member 165, which includes tension line 170 and tension ring 175. FIG. 2 illustrates tension member 165 in a relaxed state, where most of the tension line 170 is disposed within case 110. FIG. 3 shows fluid bag 310 disposed within case 110 after the tension member 165 has been moved to a position as shown in FIG. 3. Tension member 165 may additionally or alternatively include a ratchet arrangement (not shown) that enables pressure plate 155 to be withdrawn to and selectively held in the position shown in FIG. 3.

In operation, when the ratchet arrangement is engaged, the ratchet allows pressure plate 155 to move in one direction (e.g., towards a location in half 12 of case 110 where hose 150 extends from case 110). When released, the ratchet arrangement does not restrict movement of pressure plate 155. The spring bias mechanism may store potential energy in the springs 135—load the springs 135—when springs 135 are extended (or compressed) by tension member 165. When springs 135 are extended and tension member 165 is released, pressure plate 15 applies pressure to the fluid bag 310. When nozzle 145 is moved to its open position by the finger- or thumb-operated lever 140 (for example during cleaning of a wound area), the pressure from pressure plate 155 urges fluid from fluid bag 310 through the nozzle 145.

Many materials may be advantageously employed in the apparatus. Case 110 may be formed from any suitable rigid material. Examples of suitable materials include metals (e.g., aluminum, titanium, etc.), polymers (e.g., polyvinyl chloride, polyethylene, etc.), and the like. Case 110 may fit, for example, in a soldier's or hiker's first aid kit. In some embodiments, case 110 is smaller than 7 inches long by 6 inches wide by 6 inches tall. Springs 135 maybe formed from, for example, hardened steel, non-ferrous metals (e.g., phosphor bronze, titanium, etc.), beryllium copper, and the like. Pressure plate 155 may, for example, be made from metals, polymers, and the like. In some embodiments, pressure plate 155 is made from thermoplastic acrylic-polyvinyl chloride alloy. Hose 150 may, for example, be made from silicone rubber, latex, and the like. Fluid bag 310 may be made from polyvinyl chloride, ethylene vinyl acetate, copolyester ether, and the like. The fluid used in the fluid bag 310 may be, for example, saline (e.g., normal saline solution 0.90% w/v NaCl), iodine (e.g., 1% povidone-iodine solution), or other antiseptic solutions such as Pluronic F-68, and the like. The fluid in fluid bag 310 may be sterilized (e.g., by autoclaving).

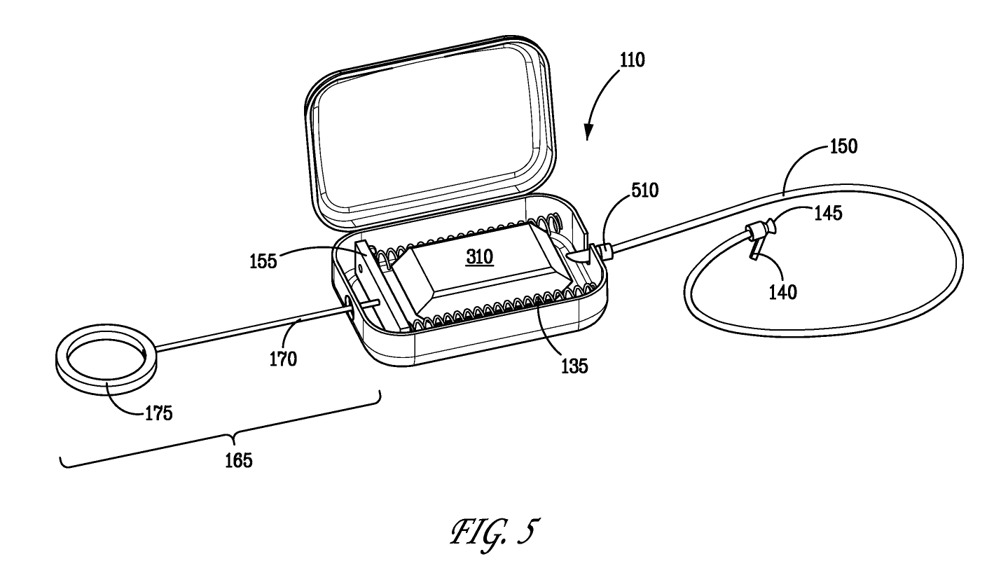

FIG. 5 is a perspective view of portable lavage apparatus 100. Portable lavage apparatus 100 may include case 110 (comprising case halves 125 and 130), pressure plate 155, tension line 170, tension ring 175, springs 135, outlet hose 150, and nozzle 145. Hose 150 may be connected to coupler or outlet 510, which may in turn be coupled to fluid bag 310. FIG. 5 illustrates the spring bias mechanism in its extended position with tension ring 175 moved away from case 110. Portable lavage apparatus 100 may be provided with a detent arrangement that enables pressure plate 155 to be held in an open position depicted in FIG. 5. The detent arrangement may also be selectively releasable, and pressure plate 155 may be urged against bag 310 to discharge fluid in fluid bag 310 through coupler or outlet 510, hose 150, and nozzle 145.

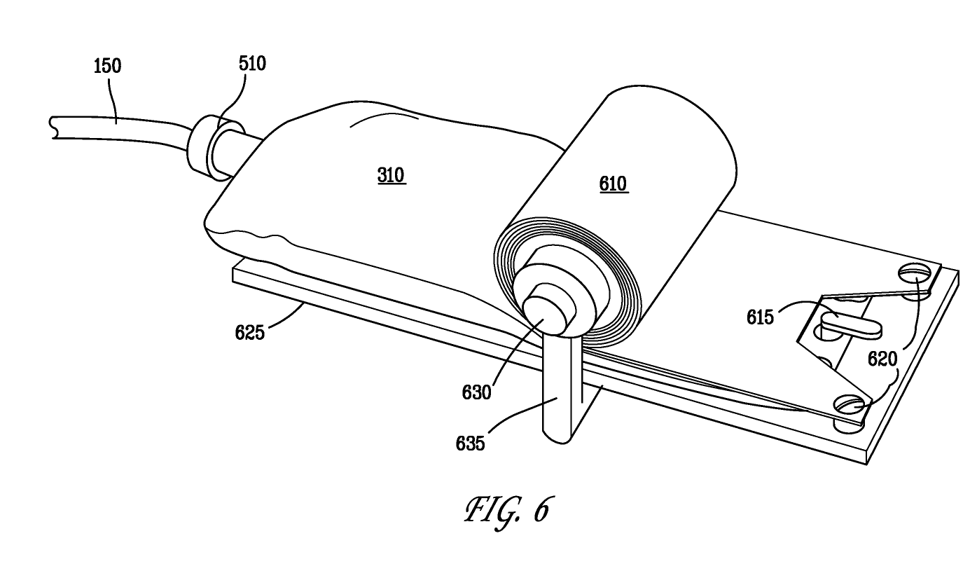

The apparatus as depicted in FIG. 6 may include a support plate 625 with the fluid bag 310 disposed thereon. Outlet 510 is coupled to the fluid bag 310 and to hose (or tube) 150. A nozzle may be disposed at the distal end of the hose 150. FIG. 6 illustrates a cylindrical wound (or coiled) spring 610 (e.g., torsion spring) which may be supported at approximately its center by support mechanism 635 which extends from support plate 625. An end of wound spring 610 is securely coupled at points 620 to support plate 625. Support plate 625 may also be coupled to lock 615. Lock 615 may be attached to an end of fluid bag 310 and hold fluid bag 310 in a (fixed) position when cylindrical coiled spring 610 is unwound. Cylindrical coiled spring 610 may be unwound by manually activating latch release 630. Latch release 630 may enable cylindrical coiled spring 610 to unwind, urging an outer surface of cylindrical coiled spring 610 against fluid bag 310, as illustrated in FIG. 6. As cylindrical coiled spring 610 urges fluid, from pressure resulting from the contact of cylindrical wound spring 610 against a surface of fluid bag 310, the fluid in fluid bag 310 may be discharged through outlet 510.

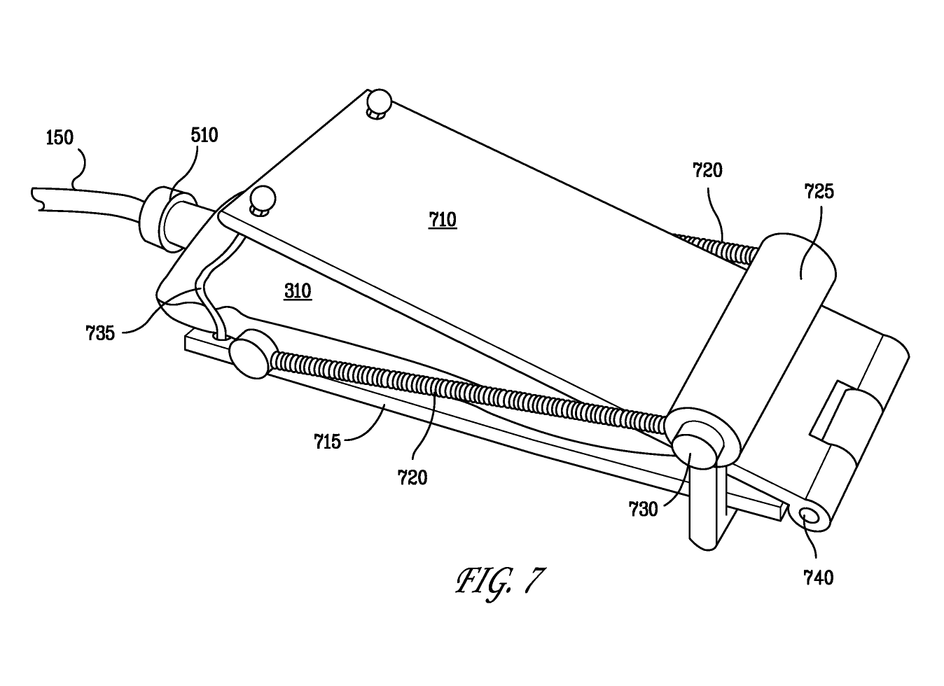

The apparatus as shown in FIG. 7 may include base plate 715, upper plate 710, and pressure member 725. Upper plate 710 is coupled to base plate 715 by hinge 740. One or more springs 720 each have an end coupled to base plate 715 and another end coupled to pressure member 725. Springs 720 apply force to pressure member 725. Fluid bag 310 is shown in a partially compressed position with upper plate 710 being urged downward by pressure member 725. Latch release button 730 may enable pressure member 725 to rotate and change position along a top surface of upper plate 710. Pressure member 725 may urge upper plate 710 toward base plate 715. When compressed between base plate 715 and upper plate 710, fluid bag 310 releases fluid. Pressure member 725 may include a roller which applies downward pressure on a top surface of upper plate 710. Latch release button 730 may control movement of pressure member 725. One or more tie wires 735, coupled to base plate 715 and upper plate 710, may limit the separation distance of ends of base plate 715 and upper plate 710 opposite hinge 740.

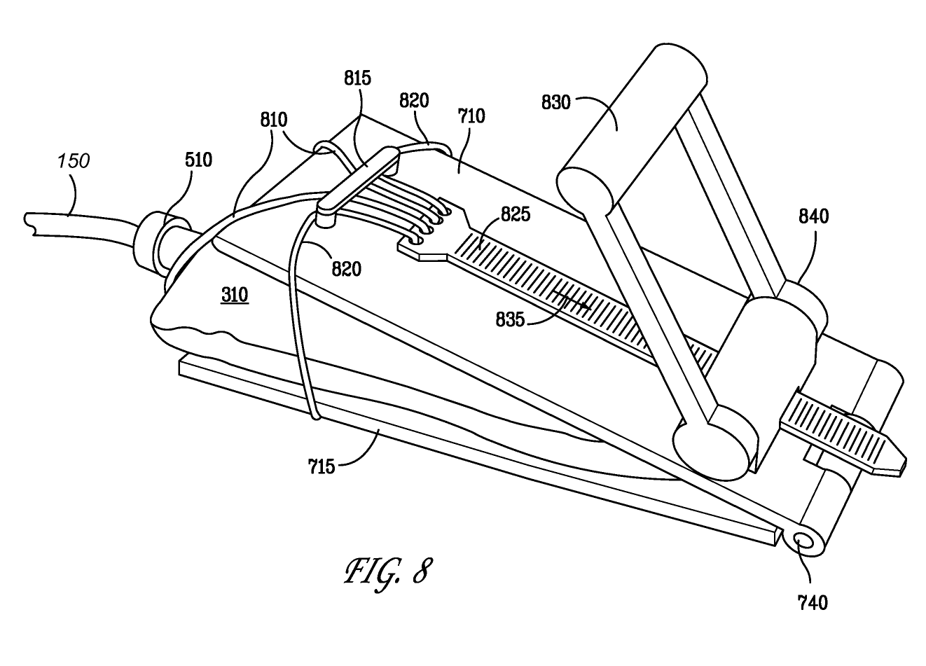

The apparatus, as depicted in FIG. 8, may also include a ratchet handle 830. Ratchet handle 830 may include base 840 which may be coupled to a surface of upper plate 710. A series of cords (or wires) 810 may be disposed between base plate 715 and upper plate 710. An end of each of cords 810 may be coupled to a surface of base plate 715 and extend about portions of fluid bag 310. Another end of each of cords 810 may be passed through guide 815 and attached to ratchet strap 825. Ratchet strap 825 may engage with a pawl arrangement (not shown) at base 840 of ratchet handle 830. Ratchet handle 830 may be moved back and forth, and when ratchet handle 830 pivots to the right in FIG. 8, ratchet handle 830 may tighten ratchet strap 825 in the direction depicted by arrow 835. As ratchet strap 825 moves in the direction depicted by arrow 835, this motion may pull cords 810, causing upper plate 710 to pivot toward base plate 715 and compress fluid bag 310.

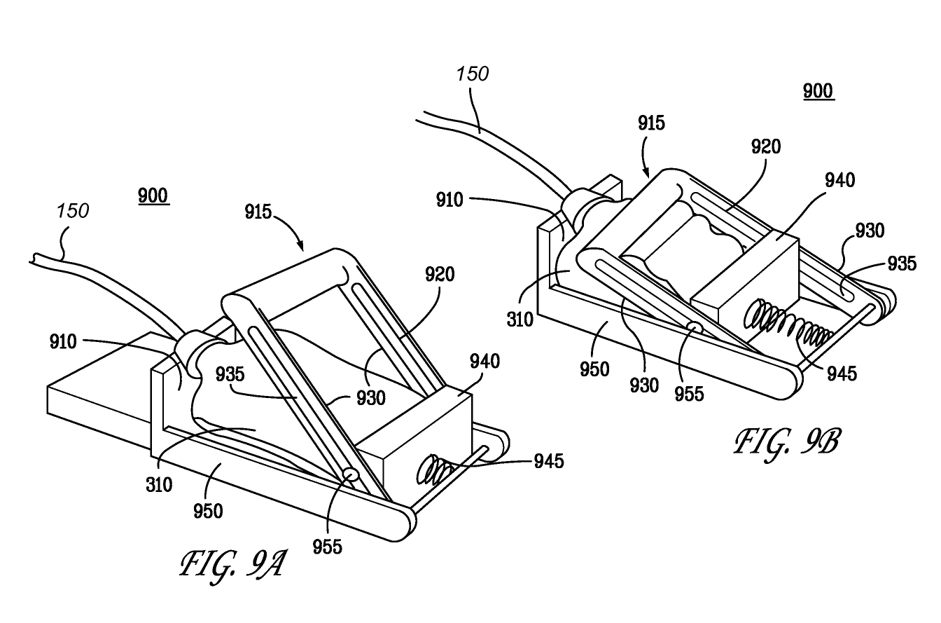

FIGS. 9A-B show separate perspective views of portable lavage apparatus 900 in two different positions. FIG. 9A depicts portable lavage apparatus 900 with pressure block 940 in an open position with spring 945 is compressed. FIG. 9B depicts portable lavage apparatus 900 with pressure block 940 in a closed position when single spring 945 is extended. Portable lavage apparatus 900 may include a base plate 950 with the fluid bag 310 disposed on base plate 950. Pivot member 915 may be coupled to base plate 950 and include side legs 930 each having respective elongated slots 920 that receive pins 955 of pressure block 940. One end of spring 945 may be coupled to base plate 950 and an opposite end of spring 945 is coupled to pressure block 940.

As pivot member 915 pivots downwardly, pressure block 940 moves along the slots 935 and applies force to fluid bag 310. As the fluid bag 310 is compressed, the fluid in fluid bag 310 flows through the outlet tubing 135.

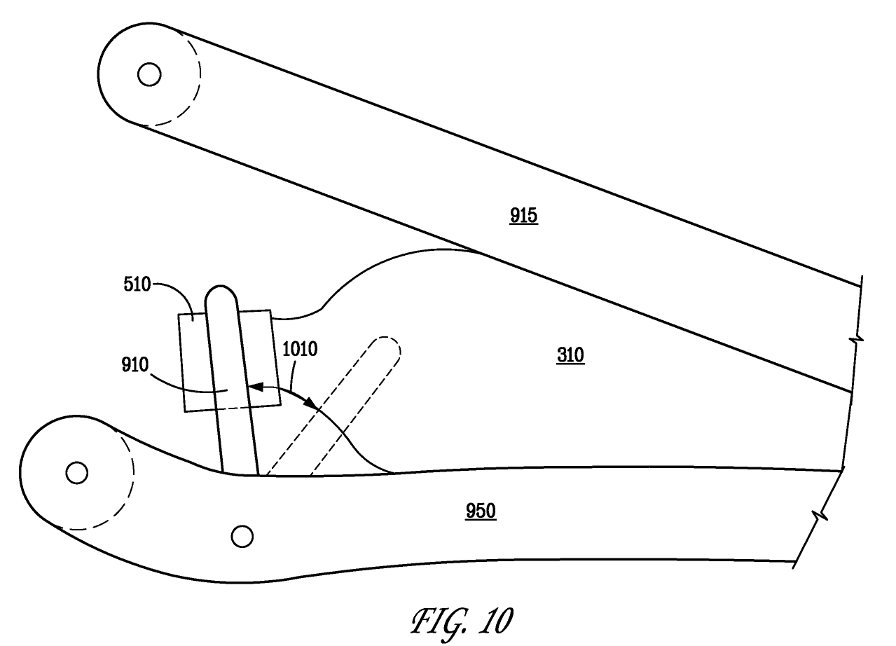

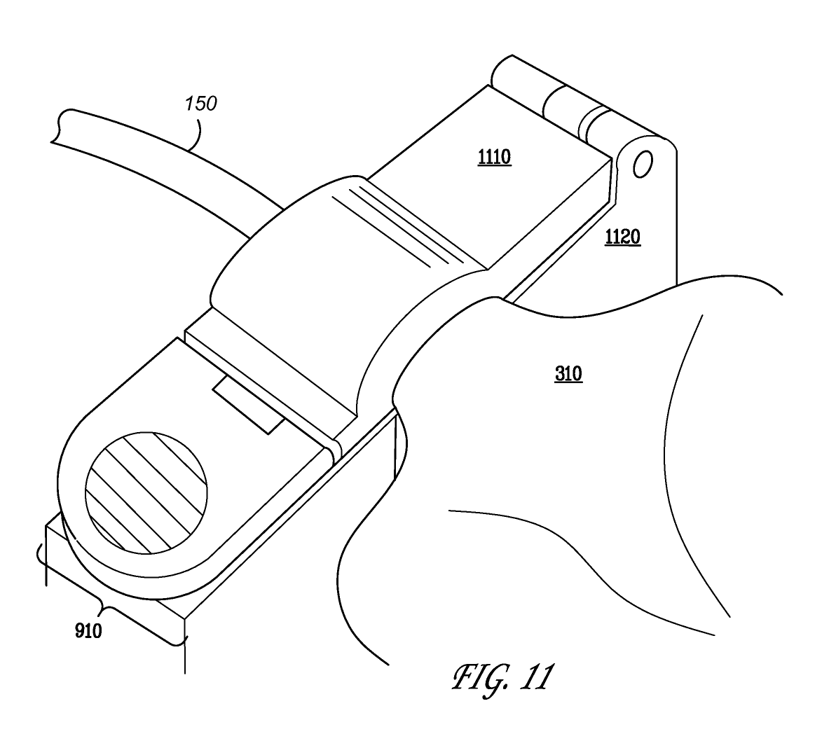

As shown in FIG. 10, a yoke 910 may be rotatably coupled to base plate 950, and may comprise upper piece 1110 and lower piece 1120 as illustrated in FIG. 11. Yoke 910 may retain outlet 510 of fluid bag 310, holding outlet 510 and thus the fluid bag 310 in a given position. Yoke 910, as illustrated in FIG. 10, may preferably fold downward in a direction shown by arrow 1010, providing a compact and relatively thin support apparatus. Upper piece 1110 of yoke 910 may be removed or pivoted away from lower piece 1120 (i.e., to an open position). When yoke 910 is in the open position, fluid bag 310 may be inserted into or removed from yoke 910. When upper piece 1110 of yoke 910 is in a closed position, yoke 910 holds fluid bag 310 in position as shown in FIG. 11.

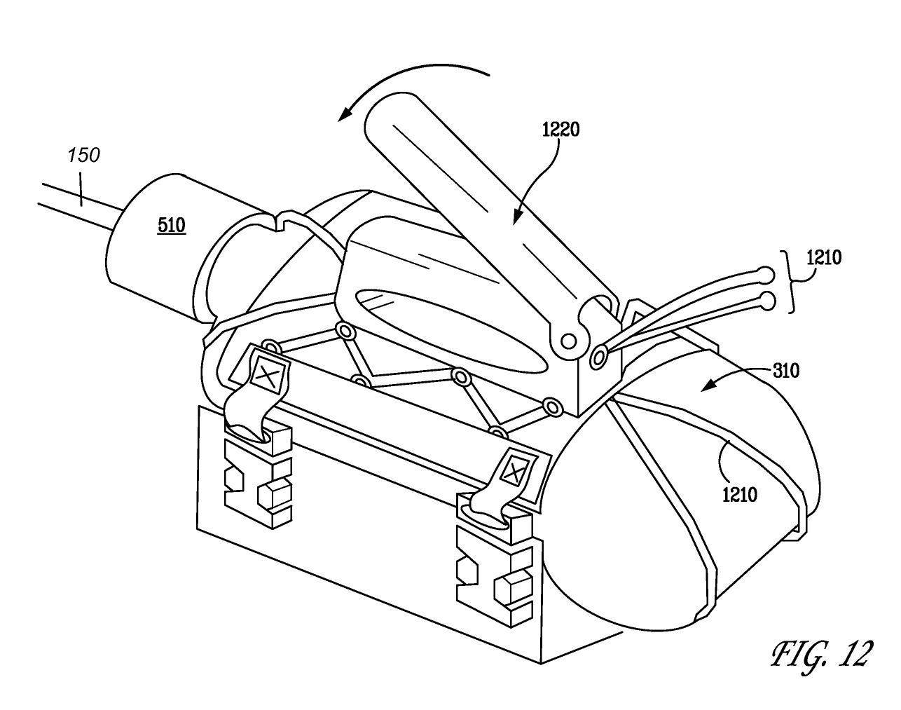

In various embodiments as illustrated in FIG. 12, the mechanism used to compress the fluid bag 310 may include one or more straps or cords 1210. Cords 1210 may be disposed about fluid bag 310 and may be tightened by manual or mechanical mechanisms. One exemplary tightening mechanism is a ratchet member 1220. Ratchet member 1220 may be used to advance cords 1210, so that cords 1210 are tightened around fluid bag 310. Cords 1210 may comprise, for example, a flexible or elastic material (e.g., nylon) and may comprise an interior core (i.e., kern) and an exterior sheath (i.e., mantle).

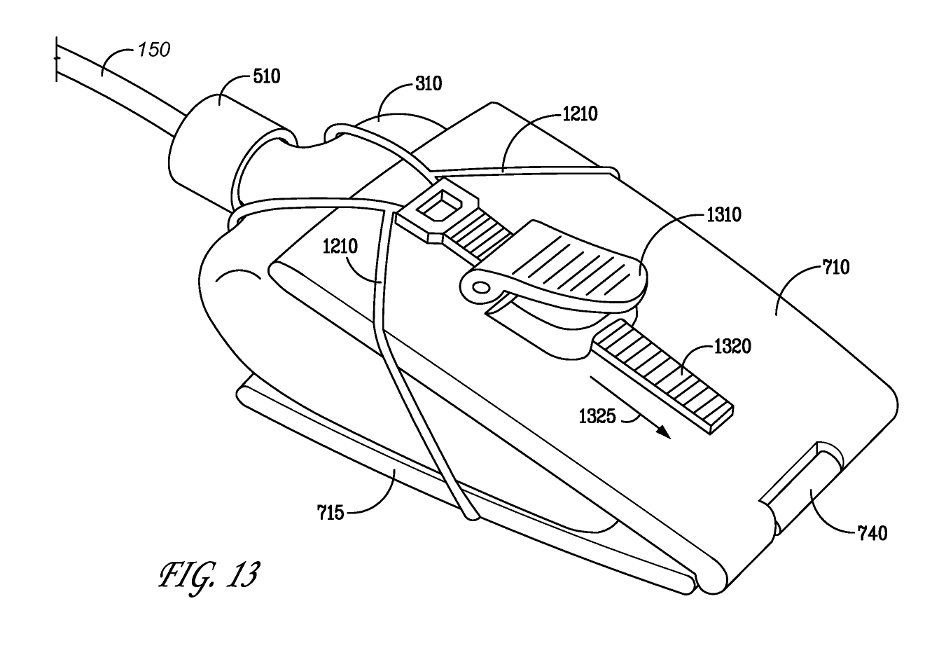

As depicted in FIG. 13, the apparatus may further include a ratcheting buckle 1310 coupled to upper plate 710. The ratcheting buckle 1310 may be activated to draw ratchet strap 1320 through ratcheting buckle 1310. Activating ratcheting buckle 1310 may pull cords 1340 and move upper plate 1340 toward base plate 715 to apply pressure to the fluid bag 310. Ratcheting strap 1320 may not be coupled to base plate 715, but may be ratcheted through a lower portion of ratcheting buckle 1310. In operation, an upper portion of ratcheting buckle 1310 may be activated by hand, foot, or knee to draw ratcheting strap 1320 in the direction indicated by arrow 1325.

An important element of first aid procedure in the field is the intravenous (IV) replacement of fluids. In some instances, the injured/wounded person may be losing fluids at such a high rate that a high pressure IV is required. So while 100 cm head pressure is adequate for low pressure IV, high pressure IV may require 300 cm head pressure or more. It will be recognized by those skilled in the art that these pressures are illustrative only. Operating conditions and requirements will determine the actual pressures that may be used.

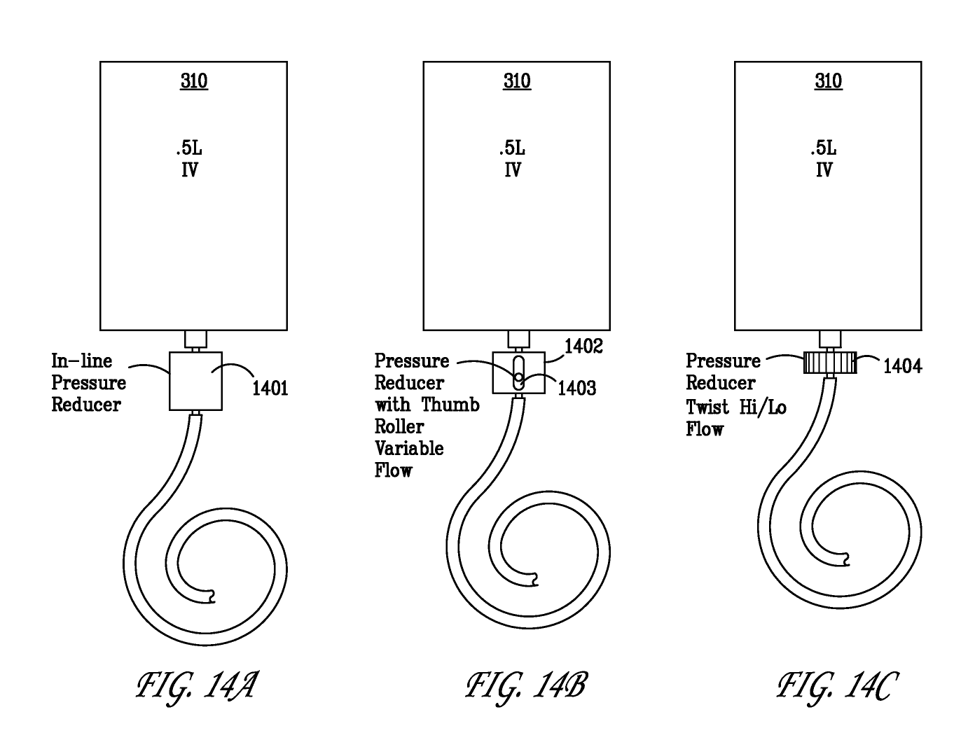

Because of the variable pressures that may be required, a pressure variation and control mechanism may be included in the fluid flow line. FIGS. 14A-C illustrate several mechanisms by which this may be accomplished. FIG. 14A shows the fluid bag 310 coupled to an in-line pressure reducer 1401. The spring bias mechanism of the lavage apparatus may still be used to apply pressure to the fluid bag 310. The in-line pressure reducer 1401 may be used to reduce the pressure to a usable level. Because the fluid in fluid bag 310 may be under pressure, fluid bag 310 does not necessarily have to be hung in an elevated position to be used.

FIG. 14B shows another example of a pressure control mechanism. The pressure reducer 1402 includes a thumb roller 1403 which allows the user to vary the pressure of the fluid flowing through dispensing hose 150 by manipulating the thumb roller 1403. FIG. 14C illustrates another pressure control mechanism, a pressure reducer 1404 that may be adjusted by manually twisting the reducer 1404.

The above examples are set forth for illustrative purposes and are not intended to limit the spirit and scope of the invention. One having skill in the art will recognize that deviations from the aforementioned examples can be created which substantially perform the same tasks and obtain similar results. For example, instead of compressing a fluid bag to release fluid, the embodiments described above may distend the fluid bag to create suction or vacuum. The vacuum may, for example, be used for negative pressure wound therapy (NPWT).

What is claimed is:

1. A portable lavage apparatus comprising:
a case;
a fluid bag disposed in the case, the fluid bag containing a fluid;
a spring bias mechanism disposed in the case; and
a hose coupled to the fluid bag.

2. The portable lavage apparatus of claim 1, wherein the spring bias mechanism comprises a pressure plate coupled to the fluid bag and one or more springs coupled to the pressure plate, the pressure plate urging the fluid from the fluid bag when moved by the springs.

3. The portable lavage apparatus of claim 2 further comprising a tension member coupled to the pressure plate and comprising a tension line to load the springs.

4. The portable lavage apparatus of claim 1, wherein the hose is substantially enclosed by the case.

5. The portable lavage apparatus of claim 1, wherein the case comprises two halves, the two halves being coupled by a hinge.

6. The portable lavage apparatus of claim 5, wherein the case further comprises a latch that couples the two halves.

7. The portable lavage apparatus of claim 1, wherein the dimensions of the case do not exceed 6 inches long by 5 inches wide by 5 inches tall.

8. The portable lavage apparatus of claim 1, wherein the fluid is an antiseptic.

9. The portable lavage apparatus of claim 1, wherein the fluid is a saline solution.

10. The portable lavage apparatus of claim 1, wherein the fluid is an iodine solution.

11. The portable lavage apparatus of claim 1, further including a pressure variation mechanism.

12. A portable lavage apparatus comprising:
a fluid bag containing a fluid;
a bias mechanism coupled to the fluid bag; and
a hose coupled to the fluid bag.

13. The portable lavage apparatus of claim 12, further comprising a support plate coupled to the fluid bag, wherein the bias mechanism comprises a cylindrical wound spring, an end of the cylindrical wound spring is coupled to the support plate, and the cylindrical wound spring is coupled to the fluid bag and urges the fluid from the fluid bag when the cylindrical wound spring unwinds.

14. The portable lavage apparatus of claim 13, further comprising a lock coupled to the support plate and the fluid bag, the lock holding the fluid bag in a fixed position when the cylindrical wound spring unwinds.

15. The portable lavage apparatus of claim 13, wherein the support plate comprises a support member.

16. The portable lavage apparatus of claim 12, further comprising a base plate coupled to an upper plate by a hinge, wherein the fluid bag is disposed between the base plate and the upper plate, the bias mechanism comprises at least one spring coupled to the base plate and to a pressure member, the pressure member applying pressure to the fluid bag when the bias mechanism is actuated.

17. The portable lavage apparatus of claim 16, wherein the pressure member comprises a roller.

18. The portable lavage apparatus of claim 16, further comprising a yoke rotatably coupled to the base plate, the yoke retaining the fluid bag.

19. The portable lavage apparatus of claim 12, further comprising a base plate coupled to an upper plate by a hinge, wherein the fluid bag is disposed between the base plate and the upper plate, and the bias mechanism comprises a ratchet handle coupled to the upper plate, a ratchet strap coupled to the ratchet handle and the upper plate, and one or more cords coupled to the ratchet strap, wherein the ratchet strap pulls the one or more cords when the ratchet handle pivots so that pressure is applied to the fluid bag.

20. The portable lavage apparatus of claim 12, further including a pressure variation mechanism.
